(19) United States
(12) Patent Application Publication
(10) Pub. No.: US 2004/0014990 A1
(43) Pub. Date: Jan. 22, 2004

(54) Preparation of maleic anhydride and catalyst for this purpose

(76) Inventors: Sebastian Storck, Mannheim (DE); Jens Weiguny, Freinsheim (DE); Mark Duda, Ludwigshafen (DE); Gerhard Cox, Bad Durkheim (DE)

Correspondence Address: KEIL & WEINKAUF, 1350 CONNECTICUT AVENUE, N.W., WASHINGTON, DC, 20036, US

(21) Appl. No.: 10/399,153

(22) PCT Filed: Oct. 26, 2001
(86) PCT No.: PCT/EP01/12445

(30) Foreign Application Priority Data
Oct. 27, 2000 (DE) 10053494.5

(51) Int. Cl. B01J 27/198; C07D 307/34

(52) U.S. Cl. 549/259; 502/208; 502/209

(57) ABSTRACT

A vanadium-, phosphorus- and oxygen-containing catalyst for the preparation of maleic anhydride by heterogeneously catalyzed gas-phase oxidation of a hydrocarbon of at least four carbon atoms has a phosphorus/vanadium ratio of from 0.9 to 1.5, comprises particles having a mean diameter of at least 2 mm and has a composition which, using CuKα radiation (λ=1.54·10−¹⁰ m), gives a powder X-ray diffraction pattern which, in the 2θ range from 10° to 70°, has a signal/background ratio of ≦10 for all diffraction lines which are attributable to a vanadium- and phosphorus-containing phase. Said catalyst is prepared and is used for the preparation of maleic anhydride.

The present invention relates to a vanadium-, phosphorus- and oxygen-containing catalyst for the preparation of maleic anhydride by heterogeneously catalyzed gas-phase oxidation of a hydrocarbon of at least four carbon atoms and a process for its preparation.

The present invention furthermore relates to a process for the preparation of maleic anhydride by heterogeneously catalyzed gas-phase oxidation of a hydrocarbon of at least four carbon atoms using the novel catalyst.

Maleic anhydride is an important intermediate in the synthesis of γ-butyrolactone, tetrahydrofuran and 1,4-butanediol, which in turn are used as solvents or, for example, are further processed to give polymers, such as polytetrahydrofuran or polyvinylpyrrolidone.

The preparation of maleic anhydride by oxidation of hydrocarbons, such as n-butane, n-butenes or benzene, over suitable catalysts has long been known. In general, vanadium-, phosphorus- and oxygen-containing catalysts (i.e. VPO catalysts) are used for this purpose. These are generally prepared as follows:

(1) synthesis of a vanadyl phosphate hemihydrate precursor (VOHPO₄·½H₂O) from a pentavalent vanadium compound (e.g. V₂O₅), a pentavalent phosphorus compound (e.g. H₃PO₄) and a reducing alcohol (e.g. isobutanol), isolation of the precipitate and drying and, if required, shaping (e.g. pelleting); and

(2) preforming to give the vanadyl pyrophosphate ((VO)₂P₂O₇) by calcination.

Variants and different embodiments of the catalyst preparation are described, for example, in U.S. Pat. No. 4,365,069, U.S. Pat. No. 4,567,158, U.S. Pat. No. 4,996,179 and U.S. Pat. No. 5,137,860.

U.S. Pat. No. 4,365,069 and U.S. Pat. No. 4,567,158 describe the calcination of the vanadyl phosphate hemihydrate precursor under air at 400° C. or 350° C.

Furthermore, U.S. Pat. No. 4,567,158 discloses a two-stage calcination in which calcination is effected first under air at from 350 to 400° C. and then under a nitrogen/steam atmosphere at from 330 to 500° C.

U.S. Pat. No. 4,996,179 describes the calcination of the catalyst precursor in an inert atmosphere at from 343 to 704° C. prior to bringing into contact with an oxygen-containing atmosphere at elevated temperatures.

U.S. Pat. No. 5,137,860 describes the preforming of the vanadyl phosphate hemihydrate precursor by calcination in an oxygen-, steam- and, if required, inert gas-containing atmosphere at up to 300° C., a subsequent temperature increase to more than 350° C. and less than 550° C. for obtaining the vanadium oxidation state and continuation of the thermal treatment under a nonoxidizing, steam-containing atmosphere having a water content of from 25 to 75 mol %.

WO 97/12674 describes the preparation of molybdenum-modified vanadyl pyrophosphate catalysts whose precursors are calcined under conditions as described above in U.S. Pat. No. 5,137,860. Finally, the catalysts are activated in an atmosphere containing air and n-butane. The catalysts contain a substantial proportion of crystalline vanadyl pyrophosphate.

EP-A 0 799 795 describes the preparation of a VPO catalyst having an X-ray diffraction pattern defined in detail, in which the catalyst precursor is calcined first in an oxygen-containing atmosphere at from 350 to 600° C. and then under an inert gas atmosphere at from 600 to 800° C. or under a hydrocarbon/air atmosphere at from 350 to 600° C. A crystalline VPO catalyst having an intensity ratio of the X-ray diffraction lines (CuKα) of intensity (2θ=23.0°) to intensity (2θ=28.5°) of from 0.4 to 0.6 is regarded as being particularly advantageous for the oxidation of n-butane to maleic anhydride.

It is an object of the present invention to provide a catalyst for the preparation of maleic anhydride by heterogeneously catalyzed gas-phase oxidation of a hydrocarbon of at least four carbon atoms which permits a higher selectivity with respect to, and a higher yield of, maleic anhydride compared with the catalysts according to the prior art, while having at least comparable activity. It is a further object of the present invention to provide a process for the preparation of said catalyst which is technically simple to carry out. It is a further object of the present invention to provide a process for the preparation of maleic anhydride by heterogeneously catalyzed gas-phase oxidation of a hydrocarbon of at least four carbon atoms using said catalyst.

We have found that this object is achieved by a catalyst for the preparation of maleic anhydride by heterogeneously catalyzed gas-phase oxidation of a hydrocarbon of at least four carbon atoms, the catalyst containing vanadium, phosphorus and oxygen, the molar phosphorus/vanadium ratio being from 0.9 to 1.5 and the catalyst comprising particles having a mean diameter of at least 2 mm, wherein, using CuKα radiation (λ=1.54·10−¹⁰ m), the composition gives a powder X-ray diffraction pattern which, in the 2θ range from 10° to 70°, has a signal/background ratio of ≦10 for all diffraction lines which are attributable to a vanadium- and phosphorus-containing phase.

The term “composition” is to be understood as meaning all components of the catalyst, including active and inactive components.

What is important in the case of the novel catalyst is that, using CuKα radiation (λ=1.54·10−¹⁰ m), the composition gives a powder X-ray diffraction pattern which, in the 2θ range from 10° to 70°, has a signal/background ratio of ≦10, preferably ≦5, particularly preferably ≦3 and very particularly preferably ≦2, in particular ≦1, for all diffraction lines which are attributable to a vanadium- and phosphorus-containing phase.

The X-ray diffraction pattern gives the intensity of the diffracted X-rays (in counts per second=cps) as a function of twice the diffraction angle 2θ. A powder sample is used for recording the powder X-ray diffraction pattern. In the present case, the particles should therefore be powdered in order to measure the catalyst. The X-ray diffraction pattern is recorded using a powder diffractometer with variable aperture and collimator measurement being effected in the reflection mode.

The signal/background ratio of the individual diffraction lines (peaks) can be determined from the powder X-ray diffraction pattern as follows:

Selection of the diffraction signal of interest.

Determination of the mean intensity of the background in the vicinity of the diffraction signal. The vicinity of the diffraction signal is to be understood as meaning ±2° in the 2θ range, starting from the 2θ value of the intensity maximum.

Determination of the intensity of the diffraction signal of interest, i.e. of the maximum value of the measured intensity of the diffraction signal. By subsequent subtraction of the mean intensity of the background in the vicinity of the diffraction signal, the background-corrected intensity of the diffraction signal is obtained.

The signal/background ratio should then be calculated as a quotient of the background-corrected intensity of the diffraction signal and the mean intensity of the background in the vicinity of the diffraction signal.

What is important in the evaluation is correct assignment of the individual diffraction lines, since the characterization with respect to the signal/background ratio relates only to those fraction lines in the 2θ range from 10° to 70° which are attributable to a vanadium- and phosphorus-containing phase. For example, the files and databases known to a person skilled in the art, for example the PDF 2 data file of the International Center for Diffraction, are suitable for this purpose.

In the case of a superposition of two diffraction lines, one diffraction line originating from a vanadium- and phosphorus-containing phase and the other diffraction line from (i) a phase not-containing vanadium, (ii) a phase not containing phosphorus or (iii) a phase containing neither vanadium nor phosphorus, that intensity fraction of the diffraction line which is attributable to a vanadium- and phosphorus-containing phase should be calculated from the remaining diffraction pattern of this phase according to the conventional methods. For calculating the signal/background ratio for this diffraction signal, this value should then be used for the intensity of the diffraction signal of interest.

Using CuKα radiation (λ=1.54·10−¹⁰ m), the composition of the novel catalyst preferably gives a powder X-ray diffraction pattern which, in the 2θ range from 10° to 70°, has a broad intensity maximum at 30°±5° in addition to the abovementioned features with respect to the signal/background ratio.

The abovementioned, novel characterization with respect to the signal/background ratio relates to all diffraction lines in the 2θ range from 10° to 70° which are attributable to a vanadium- and phosphorus-containing phase, preferably a vanadium-, phosphorus- and oxygen-containing phase. Such a phase can usually be referred to as an amorphous VPO phase or a substantially amorphous VPO phase. The term substantially amorphous VPO phase indicates that, with regard to the characterizing signal/background ratio, crystalline fractions and phases of a vanadium- and phosphorus-containing compound, for example of crystalline vanadyl pyrophosphate (VO)₂P₂O₇, may also be present.

Furthermore, the novel catalyst may additionally contain phases which are substantially free of vanadium and/or substantially free of phosphorus, regardless of the signal/background ratio of their diffraction lines in the powder X-ray diffraction pattern. The term substantially free is to be understood as meaning a content of, in each case, ≦0.1, preferably ≦0.01, % by weight in the respective phase. Said phases may be, for example, promotor-containing phases, phases of an assistant or vanadium- or phosphorus-containing phases (e.g. vanadium pentoxide or vanadium tetroxide).

A promotor is generally to be understood as meaning an additive which improves the catalytic properties of the catalyst. Examples of suitable promoters for the novel catalyst are the elements of the 1^{st} to 1^{th} group of the Periodic Table of the Elements and their compounds. If the catalyst contains promoters, they are preferably compounds of the elements cobalt, molybdenum, iron, zinc, hafnium, zirconium, lithium, titanium, chromium, manganese, nickel, copper, boron, silicon, antimony, tin, niobium and bismuth, particularly preferably molybdenum, iron, zinc, antimony, bismuth and lithium. The novel catalyst may contain one or more promoters. The total content of promoters in the prepared catalyst is in general not more than about 5, preferably not more than about 2, % by weight, calculated in each case as oxide.

An assistant is generally to be understood as meaning an additive which advantageously influences the preparation and/or the mechanical-physical properties of the catalyst. Pelleting assistants and pore formers may be mentioned as nonrestricting examples.

Pelleting assistants are generally added if the shaping of the novel catalysts is effected by means of pelleting. Pelleting assistants are as a rule catalytically inert and improve the pelleting properties of the precursor powder, an intermediate in the catalyst preparation, for example by reducing the friction and increasing the flowability. Examples of a suitable and preferred pelleting assistant is graphite. The added pelleting assistants generally remain in the activated catalyst. Typically, the content of pelleting assistant in the prepared catalyst is from about 2 to 6% by weight.

Pore formers are substances which are used for establishing a specific pore structure in the macropore range. They can be used in principle independently of the shaping method. As a rule, they are carbon-, hydrogen-, oxygen- and/or nitrogen-containing compounds which are added before the shaping of the catalyst and are predominantly removed again during the subsequent activation of the catalyst with sublimation, decomposition and/or evaporation. The prepared catalyst may nevertheless contain residues or decomposition products of the pore former.

The novel catalyst may contain the vanadium-, phosphorus- and oxygen-containing active material, for example, in pure, undiluted form as an unsupported catalyst or in a form diluted with preferably oxidic support material, as a mixed catalyst. Examples of suitable support materials for the mixed catalysts are, for example, alumina, silica, aluminosilicates, zirconium dioxide, titanium dioxide or mixtures thereof. The unsupported and mixed catalysts are preferred, the unsupported catalysts being particularly preferred.

In the case of the novel catalyst, the molar phosphorus/vanadium ratio is from 0.9 to 1.5, preferably from 0.95 to 1.2, particularly preferably from 0.95 to 1.1, in particular from 1.0 to 1.05. The oxygen/vanadium ratio is in general ≦5.5, preferably from 4 to 5.

In the novel catalyst, the average oxidation state of the vanadium is preferably from +3.9 to +4.4, particularly preferably from +4.0 to +4.3. The novel catalyst preferably has a BET surface area of from 10 to 50, particularly preferably from 15 to 30, m²/g. It preferably has a pore volume of from 0.1 to 0.5, particularly preferably from 0.1 to 0.3, ml/g. The bulk density of the novel catalyst is from 0.5 to 1.5 kg/l.

The novel catalyst comprises particles having a mean diameter of at least 2 mm, preferably at least 3 mm. The mean diameter of a particle is to be understood as meaning the mean value of the smallest and the largest dimension between two plane parallel plates.

Particles are to be understood as meaning both irregularly shaped particles and geometrically shaped particles, i.e. moldings. The novel catalyst preferably comprises moldings. Examples of suitable moldings are pellets, cylinders, hollow cylinders, spheres, extrudates, wagon wheels or extrudates. Particular shapes, for example trilobes and tristars (cf. EP-A-0 593 646) or moldings having at least one notch in the outside (cf. U.S. Pat. No. 5,168,090), are also possible.

Particularly preferably, the novel catalyst comprises moldings having a substantially hollow cylindrical structure. A substantially hollow cylindrical structure is to be understood as meaning a structure which comprises substantially a cylinder having an orifice passing through between the two lid surfaces. The cylinder is characterized by two substantially parallel lid surfaces and a lateral surface, the cross section of the cylinder, i.e. parallel to the lid surfaces, being substantially of circular structure. The cross section of the continuous orifice, i.e. parallel to the lid surfaces of the cylinder, is likewise substantially of circular structure. Preferably, the continuous orifice is concentric with respect to the lid surfaces, other spatial arrangements not being ruled out thereby.

The term substantially indicates that deviations from the ideal geometry, for example slight deformations of the circular structure, lid surfaces which are not plane parallel, flaked-off corners and edges, surface roughness or notches in the lateral surface, the lid surfaces or the inner surface of the continuous hole, are also included in the novel catalyst. With regard to the accuracy of the pelleting art, circular lid surfaces, a circular cross section of the continuous hole, parallel lid surfaces and macroscopically smooth surfaces are preferred.

The substantially hollow cylindrical structure can be described by an external diameter d₁, a height h as the distance between the two lid surfaces and a diameter d₂ of the inner hole (continuous orifice). The external diameter d₁ of the novel catalyst is preferably from 3 to 10 mm, particularly preferably from 4 to 8 mm, very particularly preferably from 5 to 6 mm. The height h is preferably from 1 to 10 mm, particularly preferably from 2 to 6 mm, very particularly preferably from 2 to 3 mm. The diameter d₂ of the continuous orifice is preferably from 1 to 8 mm, particularly preferably from 2 to 6 mm, very particularly preferably from 2 to 3 mm.

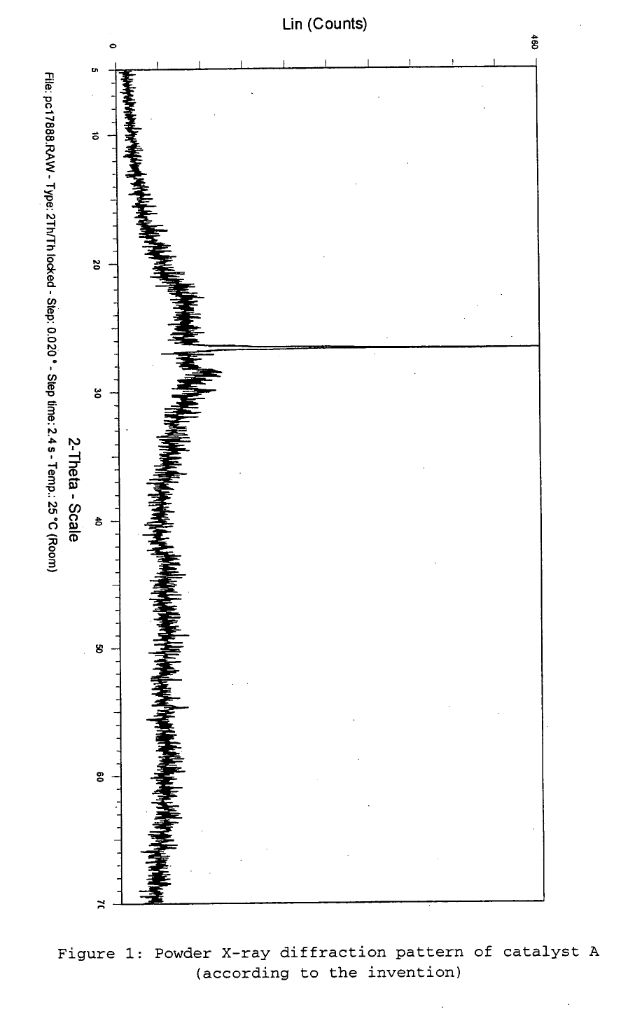

In a preferred embodiment, the hollow cylindrical catalyst comprises vanadium, phosphorus and oxygen as well as graphite as a pelleting assistant. A possible powder X-ray diffraction pattern of such a novel catalyst is shown in FIG. 1 as a nonlimiting example. A diffraction signal of strong intensity at a 2θ value of about 26.6° is clearly detectable. It is attributable to the graphite used as a pelleting assistant. Furthermore, a broad intensity maximum is detectable at about 27°. The signal/background ratio of all diffraction lines which are attributable to a vanadium- and phosphorus-containing phase is ≦0.5.

The present invention furthermore relates to a process for the preparation of a catalyst for the preparation of maleic anhydride by heterogeneously catalyzed gas-phase oxidation of a hydrocarbon of at least four carbon atoms, which comprises a catalytically active material containing vanadium, phosphorus and oxygen and in which the molar phosphorus/vanadium ratio is from 0.9 to 1.5, by (i) reaction of a pentavalent vanadium compound with a reducing agent and a phosphorus compound, (ii) isolation of the catalyst precursor formed and (iii) calcination of the catalyst precursor, wherein the calcination comprises the following steps:

(a) heating in an oxidizing atmosphere having a molecular oxygen content of ≧3% by volume and a steam content of ≦5% by volume at from 300 to 450° C.;

(b) heating in an inert gas atmosphere having a molecular oxygen content of ≦2% by volume and a steam content of ≦2% by volume at from ≦50 to 500° C. over a period which is effective for establishing in the composition a spatial atomic arrangement which, using CuKα radiation (λ1.54·10−¹⁰ m), gives a powder X-ray diffraction pattern which, in the 2θ range from 10° to 70°, has a signal/background ratio of ≦10 for all diffraction lines which are attributable to a vanadium- and phosphorus-containing phase.

The novel process for the preparation of the catalyst can be roughly divided into the three process steps

(i) reaction of a pentavalent vanadium compound with a reducing agent and a phosphorus compound;

(ii) isolation of the catalyst precursor formed; and

(iii) calcination of the catalyst precursor.

What is important in the novel process is the type and manner of the calcination of the catalyst precursor (process step (iii)), which contains the steps (a) and (b) described above. The individual process steps are described in more detail below.

(A) Calcination of the Catalyst Precursor (Process Step (iii))

The catalyst precursor contains vanadium, phosphorus and oxygen and, before the beginning of the calcination step (iii), is generally present as a finely to coarsely particulate solid, for example as powder or as moldings. Preferably, the catalyst precursor is present as moldings, particularly preferably as moldings having a mean diameter of at least 2 mm.

In step (a), the catalyst precursor is heated in an oxidizing atmosphere having a molecular oxygen content of ≧23% by volume and a steam content of ≦5% by volume at from 300 to 450° C.

The molecular oxygen content is preferably ≧5, particularly preferably ≧10, % by volume. The maximum content of molecular oxygen is in general ≦50, preferably ≦30, particularly preferably ≦25, % by volume. The steam content is preferably ≦3, particularly preferably ≦2, in particular ≦1, % by volume. In general, a mixture of oxygen and an inert gas (e.g. nitrogen or argon), a mixture of oxygen and air, a mixture of air and an inert gas (e.g. nitrogen or argon) or air is used in step (a). The use of air is preferred. It is advantageous if a certain gas exchange is ensured in the calcination furnace during step (a) so that the gases released by the catalyst precursor, for example steam, are removed and the required minimum content of molecular oxygen is maintained.

A temperature of from 300 to 400° C., particularly preferably from 325 to 390° C., is preferred in step (a). During the calcination step, the temperature may be kept constant or it may on average increase or decrease or vary. Since step (a) is generally preceded by a heating phase, the temperature will as a rule initially increase and then settle to the desired final value.

The period over which the heating in step (a) is maintained is preferably chosen in the novel process so that the resulting mean oxidation state of the vanadium is from +3.9 to +4.4, preferably from +4.0 to +4.3.

The mean oxidation state of the vanadium is determined by means of potentiometric titration. A description of the method is to be found, for example, under Determination of the mean oxidation state of the vanadiums.

Since the determination of the mean oxidation state of the vanadium during the calcination is extremely difficult for reasons relating to apparatus and time, the required period should advantageously be determined in preliminary experiments. As a rule, a measurement series in which heating is effected under defined conditions is used for this purpose, the samples being taken from the system after different times, cooled, and analyzed with respect to the mean oxidation state of the vanadium.

In general, the period in step (a) is more than 5, preferably more than 10, particularly preferably more than 15, minutes. In general, a period of not more than 2 hours, preferably not more than 1 hour, is sufficient for establishing the desired mean oxidation state. Under appropriately established conditions (for example lower range of the temperature interval and/or low content of molecular oxygen), however, a period of more than 2 hours is also possible.

In step (b), the catalyst intermediate obtained is heated in an inert gas atmosphere having a molecular oxygen content of ≦2% by volume and a steam (H₂O) content of ≦2% by volume at from 350 to 500° C. over a period which is effective for establishing in the composition a spatial atomic arrangement which, using CuKα radiation (λ=1.54·10−¹⁰ m), gives a powder X-ray diffraction pattern which, in the 2θ range from 10° to 70°, has a signal/background ratio of ≦10 for all diffraction lines which are attributable to a vanadium- and phosphorus-containing phase.

The term inert gas atmosphere is to be understood as meaning a gas atmosphere which is characterized by a molecular oxygen content of ≦2 % by volume and a steam (H₂O) content of ≦2% by volume. Preferably, the molecular oxygen content is ≦1, particularly preferably ≦0.5, % by volume. The steam content is preferably ≦1.5, in particular ≦1, % by volume. The inert gas atmosphere generally contains predominantly nitrogen and/or noble gases, for example argon, no restriction being understood thereby. Gases, for example carbon dioxide, are in principle also suitable. The inert gas atmosphere preferably contains ≧90, particularly preferably ≧95, % by volume of nitrogen.

In step (b), a temperature of from 350 to 450° C. is preferred, particularly preferably from 375 to 450° C. The temperature can be kept constant during the calcination step or it may on average increase or decrease or vary. The temperature in step (b) is preferably at the same level or higher than in step (a), particularly preferably from 40 to 80° C., in particular from 40 to 60° C., higher than in step (a).

In the novel process, the period over which the heating in step (b) is maintained is chosen so that the composition has a spatial atomic arrangement which, using CuKα radiation (λ=1.54·10−¹⁰ m), gives a powder X-ray diffraction pattern which, in the 2θ range from 10° to 70°, has a signal/background ratio of ≦10, preferably ≦5, particularly preferably ≦3 and very particularly preferably ≦2, in particular ≦1, for all diffraction lines which are attributable to a vanadium- and phosphorus-containing phase.

Since, for reasons relating to apparatus and time, it is extremely difficult to record a powder X-ray diffraction pattern during the calcination, the required period should advantageously be determined in preliminary experiments. As a rule, a measurement series in which heating is effected under defined conditions is used for this purpose, the samples being removed from the system after different times, cooled, and measured by means of the powder X-ray diffraction pattern.

In general, the period in step (b) is at least 0.5, preferably more than 1, hour and particularly preferably more than 2 hours. In general, a period of not more than 10, preferably not more than 6, hours is sufficient for establishing the desired spatial atomic arrangement.

In general, the calcination (iii) includes, as a further step (c) to be carried out after step (b), cooling in an inert gas atmosphere having a molecular oxygen content of ≦2% by volume and a steam content of ≦2% by volume to ≦300° C., preferably ≦200° C. and particularly preferably ≦150° C.

The inert gas atmosphere to be used in step (c) may differ from that in step (b) on the basis of the restrictions with regard to molecular oxygen and steam. For practical considerations, however, it is advantageous to use the same gas atmosphere as in step (b). The inert gas atmosphere to be used in step (c) should mainly suppress a change in the spatial atomic arrangement to such an extent that the required signal/background ratio of said diffraction lines in the powder X-ray diffraction pattern is maintained.

In the novel process, further steps are possible before, between and/or after the steps (a) and (b) or (a), (b) and (c). For example, changes in the temperature (heating, cooling), changes in the gas atmosphere (changeover of the gas atmosphere), further residence times, transfers of the catalyst intermediate to other apparatuses or interruptions of the total calcination process may be mentioned as further steps without having a limiting effect.

Since, as a rule, the catalyst precursor is heated to <100° C. before the beginning of the calcination, it should usually be heated before step (a). The heating can be carried out using different gas atmospheres. Preferably, the heating is carried out in an oxidizing atmosphere, as defined under step (a), or an inert gas atmosphere, as defined under step (b). A change of gas atmosphere during the heating phase is also possible. Heating in the oxidizing atmosphere which is also used in step (a) is particularly preferred, in particular under an air atmosphere.

For practical considerations, the average heating rate is in general from about 0.2 to about 10, preferably from about 0.5 to about 5, ° C./min. The average heating rate is determined by establishing the starting point and end point by the generally customary tangent method and subsequently calculating two pairs of values from these. The upper limit of the average heating rate is determined mainly by the apparatus to be used, and the lower limit by the time which is required for the total heating process and which advantageously should be within an economically expedient range. It should be pointed out explicitly that the actual heating rate, i.e. the heating rate at a specific time, may differ very greatly within the heating process. For technical reasons, the heating rate in the first half of the heating process is usually higher than in the second half. Typical values are in general from 2 to 10, preferably from 5 to 10, ° C./min for the first half and in general from 0.2 to 5° C./min for the second half.

The heating in step (b) preferably directly follows the heating of step (a), the gas atmosphere of course being changed over from an oxidizing atmosphere to an inert gas atmosphere, according to the abovementioned information. As mentioned in the above statements on step (b), the temperature of step (b) is preferably higher than that of step (a).

After step (b), cooling as described in step (c) is preferably effected.

In the novel process, the process step of calcination (iii) can be carried out in different apparatuses which are suitable for establishing the required parameters (e.g. temperature, gas atmosphere). Examples of suitable apparatuses are shaft furnaces, tray furnaces, muffle furnaces, tubular furnaces and rotary kilns.

(B) Reaction of a Pentavalent Vanadium Compound With a Reducing Agent and a Phosphorus Compound (Process Step (i))

In the preparation of the catalyst precursor, a pentavalent vanadium compound is combined with, and reacted with, a reducing agent and a phosphorus compound.

The catalyst precursor can be prepared, for example, as described in U.S. Pat. No. 5,275,996 and U.S. Pat. No. 5,641,722 or in the laid-open application WO 97/12674.

In the novel process, the pentavalent vanadium compounds used may be the oxides, the acids and the inorganic and organic salts which contain pentavalent vanadium, or mixtures thereof. The use of vanadium pentoxide (V₂O₅), ammonium metavanadate (NH₄VO₃) and ammonium polyvanadate ((NH₄)₂V₆O₁₆) is preferred, in particular vanadium pentoxide (V₂O₅). The pentavalent vanadium compounds present as a solid are used in the form of a powder, preferably in a particle range of from 50 to 500 μm. If substantially larger particles are present, the solid is comminuted and if necessary sieved before being used. Suitable apparatuses are, for example, ball mills or planetary mills.

In the novel process, the phosphorus compounds used may be both reducing phosphorus compounds, for example phosphorous acid, and pentavalent phosphorus compounds, for example phosphorus pentoxide (P₂O₅), orthophosphoric acid (H₃PO₄), pyrophosphoric acid (H₄P₂O₇), polyphosphoric acids of the formula Hₙ₊₂PₙO₃ₙ₊₁, where n ≧3, or mixtures thereof. The use of pentavalent phosphorus compounds is preferred. Usually, the content of said compounds and mixtures is stated in % by weight, based on H₃PO₄. The use of from 80 to 110% strength H₃PO₄ is preferred, particularly preferably from 95 to 110, very particularly preferably from 100 to 105, % strength H₃PO₄.

The reducing agent used may be both inorganic compounds, for example reducing phosphorus compounds (e.g. phosphorous acid), and organic compounds, for example alcohols. The use of unsubstituted or substituted, acyclic or cyclic C₁- to C₁₂-alcohols is preferred. Suitable examples are methanol, ethanol, 1-propanol, 2-propanol (isopropanol), 1-butanol, 2-butanol (sec-butanol), 2-methyl-1-propanol (isobutanol), 1-pentanol (amyl alcohol), 3-methyl-l-butanol (isoamyl alcohol), 1-hexanol, 1-heptanol, 1-octanol, 1-nonanol, 1-decanol, 1-undecanol and 1-dodecanol. 1-Butanol and 2-methyl-1-propanol (isobutanol) are particularly preferred, especially 2-methyl-1-propanol (isobutanol).

In the novel process, vanadium pentoxide is preferably used as pentavalent vanadium compound, an unsubstituted or substituted, acyclic or cyclic C₁- to C₁₂-alkanol as a reducing agent and orthophosphoric acid, pyrophosphoric acid, a polyphosphoric acid or a mixture thereof as the phosphorus compound.

The combination of the components pentavalent vanadium compound, phosphorus compound and reducing agent can be effected in the novel process in various ways. In general, the combination is carried out in the reaction apparatus suitable for the subsequent reaction, for example a stirred kettle, at from 0 to 50° C., preferably at ambient temperature. Temperature increases are possible as a result of liberation of heat of mixing.

In a preferred variant, the reducing agent is initially taken in the reaction apparatus and the pentavalent vanadium compound is added, preferably with stirring. The phosphorus compound, which, if required, may be diluted with a further portion of the reducing agent, is then added. Unless the total amount of the reducing agent has been added, the lacking portion can likewise be added to the reaction apparatus.

In another variant, the reducing agent and the phosphorus compound are initially taken in the reaction apparatus and the pentavalent vanadium compound is added, preferably with stirring.

It should be pointed out that, in addition to the above statements, a further, liquid diluent may also be added. Examples are alcohols and, in small amounts, water. The novel process is preferably carried out without the addition of a diluent.

The relative molar ratio of the phosphorus compound to be added to the pentavalent vanadium compound to be added is in general established according to the desired ratio in the catalyst precursor.

The amount of reducing agent to be added should be greater than the amount stoichiometrically required for reducing the vanadium from the oxidation state +5 to an oxidation state of from +3.5 to +4.5. If, as in the preferred variant, no liquid diluent is added, the amount of reducing agent to be added is at least such that it is possible to form with the pentavalent vanadium compound a suspension which permits thorough mixing with the phosphorus compound to be added. If alcohols are used as the reducing agent, the molar alcohol/vanadium ratio is in general from 5 to 15, preferably from 6 to 9.

Once the pentavalent vanadium compound, the phosphorus compound and the reducing agent have been combined, the suspension is heated for the reaction of said compounds and formation of the catalyst precursor. The temperature range to be chosen is dependent on various factors, in particular on the reducing effect and on the boiling point of the components. In general, a temperature of from 50 to 200° C., preferably from 100 to 200° C., is established. The volatile components, for example water or, in the case of the preferred use of an alcohol, the reducing alcohol and its degradation products, for example aldehyde or carboxylic acid, vaporize from the reaction mixture and can either be removed or partially or completely condensed and recycled. Partial or complete recycling by refluxing is preferred. Complete recycling is particularly preferred. The reaction at elevated temperature generally takes several hours and is dependent on many factors, for example on the type of components added and on the temperature. However, the properties of the catalyst precursor can also be established and influenced in a certain range by means of the temperature and the chosen duration of heating. The parameters of temperature and time can be easily optimized for an existing system by a few experiments.

If catalyst precursors promoted by the novel process are prepared, the promotor is generally added during combination of the pentavalent vanadium compound, the phosphorus compound and the reducing agent in the form of an inorganic or organic salt. Suitable promotor compounds are, for example, the acetates, acetylacetonates, oxalates, oxides or alkoxides of the abovementioned promotor metals, for example cobalt(II) acetate, cobalt(II) acetylacetonate, cobalt(II) chloride, molybdenum(VI) oxide, molybdenum(III) chloride, iron(III) acetylacetonate, iron(III) chloride, zinc(II) oxide, zinc(II) acetylacetonate, lithium chloride, lithium oxide, bismuth(III) chloride, bismuth(III) ethylhexanoate, nickel(II) ethylhexanoate, nickel(II) oxalate, zirconyl chloride, zirconium(IV) butoxide, silicon(IV) ethoxide, niobium(V) chloride and niobium(V) oxide. For further details, reference may be made to the abovementioned WO laid-open applications and US patents.

(C) Isolation of the Catalyst Precursor Formed (Process Step (ii))

After the end of the abovementioned thermal treatment in process step (i), the catalyst precursor formed is isolated, it being possible, if necessary, also to include a cooling phase and a storage or aging phase for the cooled reaction mixture prior to isolation. In the isolation, the solid catalyst precursor is separated from the liquid phase. Suitable methods are, for example, filtration, decanting or centrifuging. The catalyst precursor is preferably isolated by filtration.

In the present subdivision, intermediate steps, for example washing and drying of the catalyst precursor and, if required, also the shaping thereof, are furthermore to be assigned to process step (ii).

The catalyst precursor isolated can be further processed with or without washing. Preferably, the catalyst precursor isolated is washed with a suitable solvent in order to remove, for example, reducing agent (e.g. alcohol) still adhering or degradation products thereof. Suitable solvents are, for example, alcohols (e.g. methanol, ethanol, 1-propanol, 2-propanol), aliphatic and/or aromatic hydrocarbons (e.g. pentane, hexane, gasolines, benzene, toluene, xylenes), ketones (e.g. 2-propanone (acetone), 2-butanone, 3-pentanone), ethers (e.g. 1,2-dimethoxyethane, tetrahydrofuran, 1,4-dioxane) or mixtures thereof. If the catalyst precursor is washed, preferably 2-propanone and/or methanol and particularly preferably methanol are used.

After the isolation of the catalyst precursor or after the washing, the solid is generally dried. The drying can be carried out under various conditions. In general, it is carried out under from 0.0 (reduced pressure) to 0.1 MPa absolute (atmospheric pressure). The drying temperature is as a rule from 30 to 250° C., it being possible to use much lower temperatures in the case of drying under reduced pressure than drying under atmospheric pressure. The blanketing atmosphere which may be present during the drying may contain oxygen, steam and/or inert gases, for example nitrogen, carbon dioxide or noble gases. Drying is preferably carried out at from 1 to 30 kPa absolute and from 50 to 200° C. under an oxygen-containing or oxygen-free residual gas atmosphere, for example air or nitrogen.

In general, the dried catalyst precusor powder obtained is converted into moldings prior to the calcination (iii), even if this is not essential for the novel process. The shaping can be effected in various ways, for example by extrusion of the catalyst precursor powder converted into a paste or by pelleting. Pelleting is preferred. Suitable moldings are, for example, pellets, cylinders, hollow cylinders, spheres, strands, wagon wheels and extrudates. Pellets and hollow cylinders are preferred, in particular hollow cylinders.

Before the shaping of the catalyst precusor, it is often advantageous to mix assistants with the catalyst precusor powder. Nonlimiting examples are pelleting aids, for example graphite, and pore formers. Reference may be made here to the statements and definitions given in the description of the catalyst.

In a preferred embodiment for shaping, the catalyst precursor powder is thoroughly mixed with from about 2 to 4% by weight of graphite and precompressed in a tablet press. The precompressed particles are milled in a mill to give granules having a particle diameter of from about 0.2 to 1.0 mm and shaped into rings in a ring tablet press.

In a further embodiment for shaping, the catalyst precursor powder is thoroughly mixed with from about 2 to 4% by weight of graphite and additionally with from 5 to 20% by weight of a pore former and further processed as described above and shaped into rings.

In a preferred embodiment, the desired amounts of vanadium pentoxide powder and isobutanol are introduced into a stirred kettle and the reactor content is converted into a suspension by stirring. The desired amount of phosphoric acid, which is preferably mixed with further isobutanol, is then allowed to run into the stirred suspension. The vanadium-, phosphorus- and alcohol-containing suspension obtained is refluxed and is kept at the desired temperature for several hours. Thereafter, the reaction mixture is cooled with further stirring and is poured onto a suction filter. The catalyst precursor filtered off is then also washed with methanol and is dried at a reduced pressure of from 1 to 30, preferably from 1 to 2, kPa absolute at from 50 to 200° C., preferably from 50 to 100° C. From about 2 to 4% by weight of graphite are then mixed, as a pelleting aid, with the catalyst precursor powder, and the mixture is then pelleted in a tablet press to give pellets or hollow cylinders. The moldings obtained are then heated in an air atmosphere to a temperature of from 300 to 450° C. and are left under these conditions for a period of from about 5 minutes to not more than 2 hours to establish the desired average oxidation state of the vanadium. The air fed in up to this point is then replaced by nitrogen, the temperature is increased preferably by from 40 to 80° C. and the moldings are left under these conditions for a further from 0.5 to 10 hours until the desired spatial atomic arrangement has been established. At the end of the calcination treatment, the moldings are cooled to <100° C. under a nitrogen atmosphere.

Furthermore, a catalyst for the preparation of maleic anhydride by heterogeneously catalyzed gas-phase oxidation of a hydrocarbon of at least four carbon atoms, the catalyst containing vanadium, phosphorus and oxygen, the molar phosphorus/vanadium ratio being from 0.9 to 1.5 and said catalyst comprising particles having a mean diameter of at least 2 mm, has been found, which catalyst is obtainable by the novel process described above.

The novel catalyst permits the preparation of maleic anhydride by heterogeneously catalyzed gas-phase oxidation of a hydrocarbon of at least four carbon atoms with a higher activity and a higher selectivity with respect to, and a higher yield of, maleic anhydride than the catalysts according to the prior art.

The novel process for the preparation of the catalyst can be carried out in a technically simple manner by reacting a pentavalent vanadium compound with a reducing agent and a phosphorus compound, isolating the catalyst precursor formed and calcining the catalyst precursor under defined conditions.

The present invention furthermore relates to a process for the preparation of maleic anhydride by heterogeneously catalyzed gas-phase oxidation of a hydrocarbon of at least four carbon atoms with oxygen-containing gases, wherein the novel catalyst according to the above description is used.

In the novel process for the preparation of maleic anhydride, in general tube-bundle reactors are used. A tube-bundle reactor in turn consists of at least one reactor tube which is surrounded by a heat transfer medium for heating and/or cooling. In general, the industrially used tube-bundle reactors contain a few hundred to several tens of thousands of parallel reactor tubes.

In the novel process, suitable hydrocarbons are aliphatic and aromatic, saturated and unsaturated hydrocarbons of at least four carbon atoms, for example 1,3-butadiene, 1-butene, 2-cis-butene, 2-trans-butene, n-butane, a C₄ mixture, 1,3-pentadiene, 1,4-pentadiene, 1-pentene, 2-cis-pentene, 2-trans-pentene, n-pentane, cyclopentadiene, dicyclopentadiene, cyclopentene, cyclopentane, a C₅ mixture, hexenes, hexanes, cyclohexane and benzene. 1-Butene, 2-cis-butene, 2-trans-butene, n-butane, benzene and mixtures thereof are preferably used. The use of n-butane and n-butane-containing gases and liquids is particularly preferred. The n-butane used may originate, for example, from natural gas, from steam crackers or from FCC crackers.

The hydrocarbon is added in general under flow rate control, i.e. with continuous specification of a defined amount per unit time. The hydrocarbon may be metered in in liquid or gaseous form. Metering in liquid form with subsequent vaporization before entry into the tube-bundle reactor is preferred.

The oxidizing agents used are oxygen-containing gases, for example air, synthetic air, a gas enriched with oxygen or pure oxygen, i.e. oxygen originating from, for example, air separation. The oxygen-containing gas, too, is added with a flow rate control.

The gas to be passed through the tube-bundle reactor generally contains inert gas. Usually, the amount of inert gas at the beginning is from 50 to 95% by volume. Inert gases are all gases which do not directly contribute to the formation of maleic anhydride, for example nitrogen, noble gases,.carbon-monoxide, carbon dioxide, steam, oxygenated and nonoxygenated hydrocarbons of less than four carbon atoms (e.g. methane, ethane, propane, methanol, formaldehyde, formic acid, ethanol, acetyaldehyde, acetic acid, propanol, propionaldehyde, propionic acid, acrolein, crotonaldehyde) and mixtures thereof. In general, the inert gas is introduced into the system via the oxygen-containing gas. However, it is also possible to feed in further inert gases separately. Enrichment with further inert gases which, for example, may originate from partial oxidation of the hydrocarbons is possible by means of partial recycling of any worked-up reaction discharge.

In order to ensure a long catalyst life and a further increase in the conversion, selectivity, yield, catalyst loading and space-time yield, a volatile phosphorus compound is preferably added to the gas in the novel process. The concentration of said phosphorus compound at the beginning, i.e. at the reactor entrance, is at least 0.2 ppm by volume, i.e. 0.2·10⁻⁶ part by volume, based on the total volume of the gas at the reactor entrance, of the volatile phosphorus compounds. A content of from 0.2 to 20, particularly preferably from 0.5 to 10, ppm by volume is preferred. Volatile phosphorus compounds are to be understood as meaning all those phosphorus-containing compounds which are present in gaseous form in the desired concentration under the conditions of use. Examples are compounds of the formulae (I) and (II)

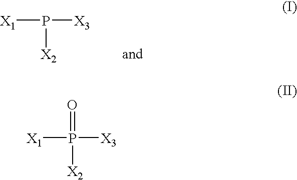

where X¹, X² and X³, independently of one another, are each hydrogen, halogen, C₁- to C₆-alkyl, C₃- to C₆-cycloalkyl, C₆- to C₁₀-aryl, C₁- to C₆-alkoxy, C₃- to C₆-cycloalkoxy or C₆- to C₁₀-aryloxy. Compounds of the formula (III)

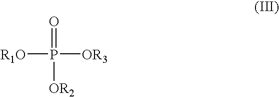

where R¹, R² and R³, independently of one another, are each hydrogen, C₁- to C₆-alkyl, C₃- to C₆-cycloalkyl or C₆- to C₁₀-aryl, are preferred. The compounds of the formula (II) in which R¹, R² and R³, independently of one another, are each C₁- to C₄-alkyl, for example methyl, ethyl, ptopyl, 1-methylethyl, butyl, 1-methylpropyl, 2-methylpropyl or 1,1-dimethylethyl, are particularly preferred. Trimethyl phosphate, triethyl phosphate and tripropyl phosphate are very particularly preferred, especially triethyl phosphate.

The novel process is generally carried out at from 350 to 480° C. Said temperature is understood as meaning the temperature of the catalyst bed which is contained in the tube-bundle reactor and would be present if the process was carried out in the absence of a chemical reaction. If this temperature is not exactly the same at all points, the term means the number average of the temperatures along the reaction zone. In particular, this means that the true temperature present at the catalyst may also be outside the stated range, owing to the exothermic nature of the oxidation reaction. The novel process is preferably carried out at from 380 to 460° C., particularly preferably from 380 to 430° C.

The novel process can be carried out at below atmospheric pressure (e.g. up to 0.05 MPa absolute) or at above atmospheric pressure (e.g. up to 10 MPa absolute). This is to be understood as meaning the pressure present in the tube-bundle reactor unit. A pressure from 0.1 to 1.0 MPa absolute is preferred, particularly preferably from 0.1 to 0.5 MPa absolute.

The novel process can be carried out in two preferred process variants, the variant involving a straight pass and the variant involving recycling. In the case of the straight pass, maleic anhydride and, if required, oxygenated hydrocarbon byproducts are removed from the reactor discharge and the remaining gas mixture is discharged and, if desired, incinerated to produce heat energy. In the case of the recycling, maleic anhydride and, if required, oxygenated hydrocarbon by-products are likewise removed from the reactor discharge and the remaining gas mixture, which contains unconverted hydrocarbon, is wholly or partly recycled to the reactor. A further variant of the recycling comprises the removal of unconverted hydrocarbon and the recycling thereof to the reactor.

In a particularly preferred embodiment for the preparation of maleic anhydride, n-butane is used as a starting hydrocarbon and the heterogeneously catalyzed gas-phase oxidation is carried out in a straight pass over the novel catalyst.

Air as the oxygen- and inert gas-containing gas is introduced into the feed unit under flow rate control. n-Butane is fed in via a pump, likewise with flow rate control but preferably in liquid form, and is vaporized in the gas stream. The ratio of the amounts of n-butane and oxygen fed in is generally established according to the exothermic nature of the reaction and the desired space-time yield and is therefore dependent, for example, on the type and amount of the catalyst. As a further component, preferably trialkyl phosphate is added, with flow rate control, as the volatile phosphorus compound to the gas stream. The volatile phosphorus compound may be added, for example, undiluted or diluted in a suitable solvent, for example water. The amount of phosphorus compound required is dependent on various parameters, for example on the type and amount of the catalyst or on the temperatures and pressures in the plant, and is to be adapted for each system.

The gas stream is passed through a static mixer for thorough mixing and through a heat exchanger for heating. The thoroughly mixed and preheated gas stream is then passed to the tube-bundle reactor in which the novel catalyst is present. The tube-bundle reactor is advantageously heated by a salt melt circulation. The temperature is established so that preferably a conversion of from 75 to 90% is reached per reactor pass.

The product gas stream originating from the tube-bundle reactor is cooled in a heat exchanger and is fed to the unit for isolating the maleic anhydride. In the preferred embodiment, the unit contains at least one apparatus for absorptive removal of the maleic anhydride and, if desired, the oxygenated hydrocarbon byproducts. Suitable apparatuses are, for example, containers which are filled with an absorption liquid and through which the cooled discharge gas is passed, or apparatuses in which the absorption liquid is sprayed into the gas stream. For further processing or for isolating the desired product, the maleic anhydride-containing solution is discharged from the plant. The remaining gas stream is likewise discharged from the plant and, if required, fed to a unit for recovering the unconverted n-butane.

The novel process using the novel catalysts permits a high hydrocarbon loading of the catalyst in combination with a high conversion owing to a high activity. The novel process furthermore permits a high selectivity, a high yield and therefore also a high space-time yield of maleic anhydride.

### EXAMPLES

Definitions

Unless stated otherwise, the quantities used in this publication are defined as follows:

$$\begin{matrix}Conversion C = \frac{{n}_{HC , reactor , in} - {n}_{HC , reactor , out}}{{n}_{HC , reactor , in}} \\ Selectivity S = \frac{{n}_{MAA , reactor , out}}{{n}_{HC , reactor , in} - {n}_{HC , reactor , out}} \\ Yield Y = C \cdot S\end{matrix}$$

X-ray Diffraction Analysis of the Catalysts

For the X-ray diffraction analysis, the catalysts were powdered and measured in an X-ray powder diffractometer of the type D5000 Theta/Theta from Siemens. The measurement parameters were as follows:

| | |
|---|---|
| Circle diameter | 435 mm |
| X-rays | CuKα (λ = 1.54 · 10−¹⁰ m) |
| Tube voltage | 40 kV |
| Tube current | 30 mA |
| Aperture | variable V20 |
| Collimator | variable V20 |
| Secondary monochromator | Graphite |
| Monochromator aperture | 0.1 mm |
| Scintillation counter | |
| Detector aperture | 0.6 mm |
| Step width | 0.02° 2θ |
| Step mode | continuous |
| Measuring time | 2.4 s/step |
| Measuring speed | 0.5° 2θ/min |

The signal/background ratio of the diffraction lines of the powder X-ray diffraction pattern was determined as described in the text.

Determination of the Average Oxidation State of the Vanadium

The average oxidation state of the vanadium was determined by means of potentiometric titration according to the method described below.

For the determination, in each case from 200 to 300 mg of the sample are added, under an argon atmosphere, to a mixture of 15 ml of 50% strength sulfuric acid and 5 ml of 85% strength phosphoric acid and dissolved with heating. The solution is then transferred to a titration vessel which is equipped with two Pt electrodes. The titrations are carried out in each case at 80° C.

First, a titration is carried out with 0.1 molar potassium permanganate solution. If two steps are obtained in the potentiometric curve, the vanadium was present in an average oxidation state of from +3 to less than +4. If only one step is obtained, the vanadium was present in an oxidation state of from +4 to less than +5.

In the first-mentioned case (two steps/+3≦V_{OX}<+4), the solution contains no V⁵⁺, i.e. all the vanadium was detected titrimetrically. The amount of V³⁺ and V⁴⁺ is calculated from the consumption of the 0.1 molar potassium permanganate solution and the position of the two steps. The weighted mean then gives the average oxidation state.

In the second-mentioned case (one step/+4≦V_{OX}<+5), the amount of V⁴⁺ can be calculated from the consumption of the 0.1 molar potassium permanganate solution. By subsequent reduction of all the V⁵⁺ of the resulting solution with a 0.1 molar ammonium iron(II) sulfate solution and further oxidation with 0.1 molar potassium permanganate solution, the total amount of vanadium can be calculated. The difference between the total amount of vanadium and the amount of V⁴⁺ gives the amount of V⁵⁺ originally present. The weighted mean then gives the average oxidation state.

Experimental Unit

The experimental unit was equipped with a feed metering unit and an electrically heated reactor tube. The reactor tube length was 30 cm and the internal diameter of the reactor tube was 11 mm. In each case 12 g of catalyst in the form of chips having a particle size of from 0.7 to 1.0 mm were mixed with the same volume of inert material (steatite balls) and were introduced into the reactor tube. The remaining empty volume was filled with further inert material (steatite balls). The reactor was operated by the straight pass method. The reactor pressure was 0.1 MPa absolute. The oxidation gas used was air. n-Butane was vaporized and was metered in gaseous form with flow rate control. The experimental unit was operated at a GHSV of 2000 h⁻¹, an n-butane concentration of 2.0% by volume and a water content of 1.0% by volume. The product gas formed was analyzed by gas chromatography.

### Example 1

(Catalyst A, According to the Invention)

Preparation of the catalyst precursor:

11.8 kg of 100% strength orthophosphoric acid were dissolved in 150 1 isobutanol with stirring in a 240 1 stirred kettle and then 9.09 kg of vanadium pentoxide powder having a mean particle size of 120 μm (manufacturer GfE, Nuremberg, Germany) were added with further stirring. The suspension was refluxed for 16 hours and then cooled to room temperature. The resulting precipitate was filtered off and was dried overnight at 150° C. under reduced pressure. The dried powder was then heated at from 260 to 270° C. under an air atmosphere in a muffle furnace. The heated powder was thoroughly mixed at room temperature with 3% by weight of graphite and pelleted to give 5 mm×3 mm×2 mm hollow cylinders (external diameter×height×diameter of the inner hole).

Calcination:

50 g of the hollow cylinder were heated under an air atmosphere (continuous feed of 50 1 (S.T.P.)/h) in a muffle furnace to 250° C. at a heating rate of 7° C./min and then to 385° C. at a heating rate of 2° C./min and were left under these conditions for 10 minutes. Thereafter, the atmosphere was changed over to a nitrogen inert gas atmosphere by closing the air supply and adding nitrogen (feed of 50 1 (S.T.P.)/h, O₂ content ≦1% by volume and H₂O content ≦1% by volume) . Under the inert gas atmosphere established, heating was effected to 425° C. and these conditions were maintained for 3 hours. Finally, cooling to room temperature was effected.

Characterization of the Catalyst:

The catalyst obtained could be characterized by a molar phosphorus/vanadium ratio of 1.05, an average oxidation state of the vanadium of +4.15 and a BET surface area 17 m²/g. In the 2θ range from 10° to 70°, the powder X-ray diffraction pattern showed a broad intensity maximum at 27° and a signal/background ratio of ≦0.5 for all diffraction lines, with the exception of the diffraction line caused by the graphite at a 2θ value of about 26.6°. The X-ray powder diffraction pattern is shown in FIG. 1.

Catalytic Test:

The catalytic test was carried out in an experimental unit under the stated conditions at 400° C. A conversion of 85.3% and a selectivity of 69.3% were achieved. The yield obtained was 59.1%.

### Example 2

### Catalyst B, Comparative Example

Preparation of the Catalyst Precursor:

The preparation of the catalyst precursor, including the shaping, was effected analogously to Example 1.

Calcination:

The moldings were now heated under air in a muffle furnace to 250° C. at a heating rate of 7.5° C./min and then to 285° C. at a heating rate of 2° C./min and were left at this temperature for 10 minutes. Thereafter, the gas atmosphere was changed over from air to nitrogen/steam (molar ratio 1:1), heated to 425° C. and left under these conditions for 3 hours. Finally, cooling to room temperature was effected under nitrogen.

Characterization of the Catalyst:

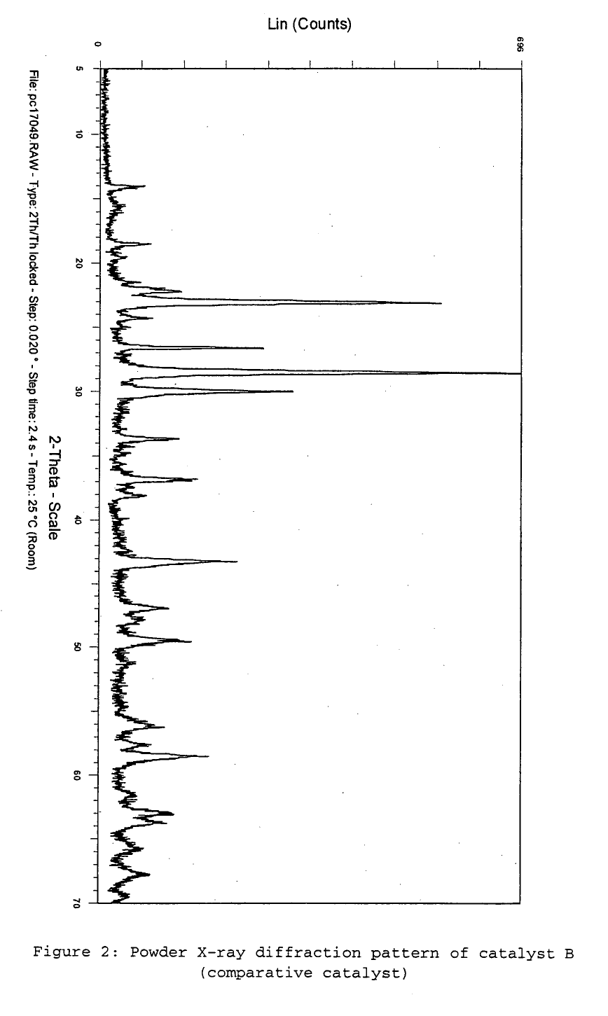

The catalyst obtained could be characterized by a molar phosphorus/vanadium ratio of 1.04, a mean oxidation state of the vanadium of +4.18 and a BET surface area of 19 m²/g. The powder X-ray diffraction pattern is shown in FIG. 2. An evaluation of the line pattern showed that the catalyst substantially comprised crystalline vanadyl pyrophosphate (VO)₂P₂O₇, the line of strongest intensity at a 2θ value of 28.5° having a signal/background ratio of 17.

Catalytic Test:

The catalytic test was carried out in an experimental unit under the stated conditions at 410° C. A conversion of 84.5% and a selectivity of 66.0% were achieved. The yield obtained was 55.8%.

Examples 1 and 2 show that, even at a temperature 10° C. lower, the novel catalyst leads to a relative conversion about 1% higher and a relative maleic anhydride yield about 6% higher.

We claim:

1. A catalyst for the preparation of maleic anhydride by heterogeneously catalyzed gas-phase oxidation of a hydrocarbon of at least four carbon atoms, the catalyst containing vanadium, phosphorus and oxygen, the molar phosphorus/vanadium ratio being from 0.9 to 1.5 and said catalyst comprising particles having a mean diameter of at least 2 mm, wherein, using CuKα radiation (λ=1.54·10−¹⁰ m), the composition gives a powder X-ray diffraction pattern which, in the 2θ range from 10° to 70°, has a signal/background ratio of ≦10 for all diffraction lines which are attributable to a vanadium- and phosphorus-containing phase.

2. A catalyst as claimed in claim 1, wherein, using CuKα radiation (λ=1.54·10−¹⁰ m), the composition gives a powder X-ray diffraction pattern which, in the 2θ range from 10° to 70°, has a signal/background ratio of ≦3 for all diffraction lines which are attributable to a vanadium- and phosphorus-containing phase.

3. A catalyst as claimed in either of claims 1 and 2, wherein the molar phosphorus/vanadium ratio is from 1.0 to 1.05.

4. A catalyst as claimed in any of claims 1 to 3, which contains a pelleting aid.

5. A catalyst as claimed in any of claims 1 to 4, wherein the average oxidation state of the vanadium is from +3.9 to +4.4, the BET surface area is from 10 to 50 m²/g, the pore volume is from 0.1 to 0.5 ml/g and the bulk density is from 0.5 to 1.5 kg/l.

6. A catalyst as claimed in any of claims 1 to 5, which comprises moldings having a substantially hollow cylindrical structure.

7. A process for the preparation of a catalyst for the preparation of maleic anhydride by heterogeneously catalyzed gas-phase oxidation of a hydrocarbon of at least four carbon atoms, which comprises a catalytically active material containing vanadium, phosphorus and oxygen and in which the molar phosphorus/vanadium ratio is from 0.9 to 1.5, by (i) reaction of a pentavalent vanadium compound with a reducing agent and a phosphorus compound, (ii) isolation of the catalyst precursor formed and (iii) calcination of the catalyst precursor, wherein the calcination comprises the following steps:
(a) heating in an oxidizing atmosphere having a molecular oxygen content of ≧3% by volume and a steam content of ≦5% by volume at from 300 to 450° C.;
(b) heating in an inert gas atmosphere having a molecular oxygen content of ≦2% by volume and a steam content of ≦2% by volume at from 350 to 500° C. over a period which is effective for establishing in the composition a spatial atomic arrangement which, using CuKα radiation (λ=1.54·10−¹⁰ m), gives a powder X-ray diffraction pattern which, in the 2θ range from 10° to 70°, has a signal/background ratio of ≦10 for all diffraction lines which are attributable to a vanadium- and phosphorus-containing phase.

8. A process as claimed in claim 7, wherein the heating in step (a) is carried out over a period which is effective for establishing an average oxidation state of the vanadium of from +3.9 to +4.4.

9. A process as claimed in either of claims 7 and 8, wherein the calcination contains as a further step to be carried out after step (b):
(c) cooling in an inert gas atmosphere having a molecular oxygen content of ≦2% by volume and a steam content of ≦2% by volume to ≦300° C.

10. A process as claimed in any of claims 7 to 9, wherein the pentavalent vanadium compound used is vanadium pentoxide, the reducing agent used is an unsubstituted or substituted acyclic or cyclic C₁- to C₁₂-alkanol and the phosphorus compound used is orthophosphoric acid, pyrophosphoric acid, a polyphosphoric acid or a mixture thereof.

11. A catalyst for the preparation of maleic anhydride by heterogeneously catalyzed gas-phase oxidation of a hydrocarbon of at least four carbon atoms, the catalyst containing vanadium, phosphorus and oxygen, the molar phosphorus/vanadium ratio being from 0.9 to 1.5 and said catalyst comprising particles having a mean diameter of at least 2 mm, obtainable by a process as claimed in any of claims 7 to 10.

12. A process for the preparation of maleic anhydride by heterogeneously catalyzed gas-phase oxidation of a hydrocarbon of at least four carbon atoms with oxygen-containing gases, wherein a catalyst as claimed in any of claims 1 to 6 or 11 is used.

13. A process as claimed in claim 12, wherein the heterogeneously catalyzed gas-phase oxidation is carried out in a tube-bundle reactor at from 350 to 480° C. and from 0.1 to 1.0 MPa absolute.

14. A process as claimed in either of claims 12 and 13, wherein the hydrocarbon used is n-butane.

15. A process as claimed in any of claims 12 to 14, wherein the heterogeneously catalyzed gas-phase oxidation is carried out in the presence of a volatile phosphorus compound.
